(19) United States
(12) Patent Application Publication
(10) Pub. No.: US 2017/0218458 A1
(43) Pub. Date: Aug. 3, 2017

(54) Method for Detecting Methylated DNA

(71) Applicant: AnchorDx Medical Co.,Ltd., Guangzhou (CN)

(72) Inventors: Jian-Bing FAN, Guangzhou (CN); Xuyu CAI, Guangzhou (CN); Yangbin GAO, (CN)

(21) Appl. No.: 15/424,748

(22) Filed: Feb. 3, 2017

(30) Foreign Application Priority Data
Feb. 3, 2016 (CN) 201610077986.1

(51) Int. Cl. C12Q 1/68

(52) U.S. Cl. CPC C12Q 1/6886; C12Q 1/6806; C12Q 1/6874; C12Q 1/6855; C12Q 2600/154; C12Q 2600/118

(57) ABSTRACT

The present application discloses a method of detecting the methylation of specific genome regions in a DNA fragment from a sample containing DNA. The method includes treating the DNA fragment containing specific genome regions with sodium bisulfite and obtaining single-strand DNA fragment; attaching an adapter to one or both ends of the single-strand DNA fragment; optionally cyclizing the adapter-attached single-strand DNA fragment; preparing the single-strand DNA fragment with attached adapter into a DNA sequencing library containing the specific genome regions; sequencing the DNA sequencing library to identify the sequence of the single-strand DNA fragment. The present application also discloses a kit for detecting the methylation of specific genome regions in a DNA fragment from a sample containing DNA, and the use of single strand ligating agent in preparing a kit for detecting the methylation of specific genome regions in a DNA fragment.

### CROSS REFERENCE TO RELATED APPLICATIONS

The present application claims priority to Chinese Patent Application No. 201610077986.1, filed on Feb. 3, 2016, the disclosure of which is hereby incorporated by reference in its entirety.

### FIELD OF THE INVENTION

The present invention relates to the method of detecting the DNA methylation of specific genome regions.

### BACKGROUND OF THE INVENTION

In eukaryotic DNA, methylation can occur to some of the cytosine, resulting in methylated DNA. Studies showed that DNA methylation may have significant influence on the functions of DNA. In particular, DNA methylation may result in alteration in chromatin structure, DNA conformation, DNA stability and the way of interaction between DNA and proteins, thereby regulating gene expression. For example, methylation in the promoter sequence of a gene usually inhibits the expression of the gene. Some studies found that DNA methylation is necessary for the normal development of organism. Other studies reported that DNA methylation is related to genomic imprinting, X chromosome inactivation, and inactivation of repetitive elements. In particular, it is found in some studies that DNA methylation is relevant to the formation of tumors.

In the promoter region or at the ends of a gene, there are usually some regions rich in “CG” nucleotide pairs (wherein C is followed immediately by G, the two nucleotides connected using a phosphodiester bond, and therefore named “CpG”). In mammalian DNA, 60%˜90% of CpG sequences are methylated (Ehrlich et al, 1982, Amount and distribution of 5-methyl-cytosine in human DNA from different types of tissues or cells, Nucleic Acids Research 10(8):2709-21). Some sections in the expression regulation region of a gene are rich in CpG (the CpG sequence appears at a frequency exceedingly higher than average), and such sections are called CpG islands. Studies show that in tumor cell DNA, significantly increased methylation in CpG islands which under normal situations are not methylated or slightly methylated, is observed in expression regulation regions of some of the genes, which inhibits the expression of said genes (e.g., tumor suppression genes). On the other hand, decreased methylation in CpG islands is observed in expression regulation regions of some of the genes in the tumor cell DNA, which results in abnormal expression of these genes (e.g., oncogenes). Therefore, important information about tumors can be obtained through the study on methylation of specific genomic regions.

In human body, a tumor cell may release its genomic DNA into the blood due to causes such as apoptosis and immune response. Normal tissues may also release normal genomic DNA into the blood. These DNA present in plasma are jointly called Cell-free DNA (cfDNA), wherein those from tumor cells are called Circulating Tumor DNA (ctDNA). Detection of methylated DNA in ctDNA is very helpful for early diagnosis of tumors. However, since the abundance of ctDNA in cfDNA is extremely low, it requires a highly sensitive method for detecting methylated DNA in ctDNA. Therefore, a method of detecting methylation of specific genome regions in a DNA fragment with great sensitivity and accuracy is in need.

### SUMMARY OF THE INVENTION

One aspect of the present invention provides a method of detecting methylation within specific genome regions in a DNA fragment from a sample containing DNA. In some embodiments, such method comprises steps of: treating the DNA fragment containing the specific genome regions with sodium bisulfite, wherein the treatment with sodium bisulfite converts unmethylated cytosine in the DNA fragment into uracil, and obtaining a single-strand DNA fragment; attaching an adapter to one end or both ends of the single-strand DNA fragment; preparing a DNA sequencing library containing the specific genome regions by amplifying the adapter-attached single-strand DNA fragment with an index primer; and sequencing the DNA sequencing library to determine sequence of the single-strand DNA fragment.

In some embodiments, the DNA fragment has completely become single-strand DNA fragments after sodium bisulfite treatment. In some embodiments, the DNA fragment has not completely become single-strand DNA fragments after sodium bisulfite treatment, and the method further comprises denaturing the sodium bisulfite treated DNA fragment to obtain DNA fragments that are completely single-strand.

In some embodiments, the adapter is attached to both ends of the single-strand DNA fragment using a single-strand DNA ligase. In some embodiments, the adapter is attached to the 3′ end of the single-strand DNA fragment using a single-strand DNA ligase, and after linear amplification of the single-strand DNA fragment with adapter attached on the 3′ end, an adapter is attached to the 3′ end of the amplification product of the single-strand DNA fragment with adapter attached on the 3′ end (corresponding to the 5′ end of the original template single-strand DNA fragment) using a single-strand DNA ligase.

In some embodiments, the adapter comprises a DNA molecular tag. In some embodiments, the index primer comprises respectively entire or part of the sequence which is identical or complementary to the sequence of each adapter. In some other embodiments, the adapter is bisulfite-treatment resistant. In some embodiments, the method further comprises treating the DNA fragment with a type II restriction endonuclease before the step of treating the DNA fragment with sodium bisulfite. In some embodiments, the type II restriction endonuclease is selected from the group consisting of HpaII/MspI, SmaI/XmaI, BamHI, HpaII.

In some embodiments, the steps of preparing the adapter-attached single-strand DNA fragment into a DNA sequencing library containing specific genome regions comprises amplification of the adapter-attached single-strand DNA fragment using an index primer. In some embodiments, the amplification is exponential amplification or linear amplification, and the index primer comprises respectively entire or part of the sequence which is identical or complementary to the sequence of each adapter.

In some embodiments, the method further comprises: cyclizing the adapter-attached single-strand DNA fragment before the preparation of DNA sequencing library. In some embodiments, the adapter-attached single-strand DNA fragment is cyclized using a highly efficient cyclization agent. In some embodiments, the highly efficient cyclization agent is a single-strand DNA circligase. In some embodiments, the single-strand DNA circligase is CircLigase. In some embodiments, the method comprises phosphorylating the 5′ end and dephosphorylating the 3′ end of the DNA fragment before the step of cyclizing the single-strand DNA fragment. In some embodiments, the steps of preparing the DNA sequencing library comprises: amplifying the cyclized single-strand DNA fragment with the index primer after the step of cyclizing the adapter-attached single-strand DNA fragment, and obtaining the DNA sequencing library comprising a DNA amplification product containing the specific genome regions. In some embodiments, the amplification step uses inverse PCR amplification. In some embodiments, the amplification step uses rolling circle amplification.

In some embodiments, the sequencing step comprises using high throughput DNA sequencing technology to determine the DNA sequence of the DNA sequencing library. In some embodiments, the step of preparing the DNA sequencing library further comprises: enriching specific amplification product using an oligonucleotide probe after the amplification step.

In some embodiments, the DNA fragment is cleaved to obtain cleaved DNA fragments with sizes of 0.1-5 kb, 0.1-1 kb, 1-2 kb, 2-3 kb, 3-4 kb, 4-5 kb, 0.2-0.4 kb, 0.5-1 kb or 0.1-0.5 kb before the step of treating the DNA fragment with sodium bisulfite, wherein the cleaved DNA fragments contains the specific genome regions.

In some embodiments, the DNA contained in the sample is cell-free DNA. In some embodiments, the cell-free DNA comprises circulating tumor DNA.

Another aspect of the present invention provides a kit for detecting methylation within specific genome regions in a DNA fragment from a sample containing DNA. In some embodiments, the kit comprises: sodium bisulfite, which can be used to convert unmethylated cytosine in the DNA fragment into uracil; a single-strand DNA ligating agent, which can be used to ligate a single-strand DNA fragment with an adapter; and a sequencing agent. In some embodiments, the single-strand DNA ligating agent comprises the adapter, a single-strand DNA ligase, a reacting solution. In some embodiments, the kit further comprises a library preparing agent, which can be used to amplify the single-strand DNA fragment containing specific genome regions, the library preparing agent comprises an index primer. In some embodiments, the library preparing agent further comprises an amplification agent, which, in some embodiments, comprises a DNA polymerase, a reaction solution, and dNTPs. In some embodiments, the kit further comprises linear amplification agent, wherein the linear amplification agent comprising a linear amplification primer, and the linear amplification primer comprises entire or part of complementary sequence of the adapter attached to one end of the single-strand DNA fragment. In some embodiments, the kit further comprises a cyclization agent, which can cyclize a single-strand DNA fragment. In some embodiments, the kit further comprises a cleaving agent, which can be used to cleave the DNA fragment into cleaved DNA fragments with size of 0.1-5 kb, 0.1-1 kb, 1-2 kb, 2-3 kb, 3-4 kb, 4-5 kb, 0.2-0.4 kb, 0.5-1 kb or 0.1-0.5 kb before sodium bisulfite treatment.

In some embodiments, the cyclizing agent is a highly efficient cyclization agent. In some embodiments, the highly efficient cyclization agent is a single-strand DNA circligase. In some embodiments, the single-strand DNA circligase is CircLigase. In some embodiments, the cyclization agent further comprises T4 PNK, which can be used for the phosphorylation on the 5′ end and the dephosphorylation on the 3′ end of a DNA fragment. In some embodiments, the library preparing agent comprises an agent used in inverse PCR amplification or an agent used in rolling circle amplification.

Yet another aspect of the present invention provides the use of a single-strand ligating agent in preparation of a kit for detecting methylation within specific genome regions in a DNA fragment from a sample containing DNA, wherein the detection comprises the following steps: treating the DNA fragment containing specific genome regions with sodium bisulfite, wherein the treatment with sodium bisulfite converts unmethylated cytosine in the DNA fragment into uracil and obtaining a single-strand DNA fragment; attaching adapter to one end or both ends of the single-strand DNA fragment; preparing a DNA sequencing library containing the specific genome regions by amplifying the adapter attached single-strand DNA fragment with index primer; and sequencing the DNA sequencing library. In some embodiments, the adapter is attached to both ends of the single-strand DNA fragment using a single-strand DNA ligase. In some embodiments, an adapter is attached to the 3′ end of the single-strand DNA fragment using a single-strand DNA ligase, and after linear amplification of the single-strand DNA fragment with adapter attached on the 3′ end, an adapter is attached to the 3′ end of the amplification product of the single-strand DNA fragment with adapter attached on the 3′ end using a single-strand DNA ligase.

In some embodiments, the detection further comprises cyclizing the single-strand DNA fragment, and after the cyclization step, amplifying the cyclized single-strand DNA fragment using an index primer to prepare a DNA sequencing library containing the specific genome regions.

In some embodiments, the detection further comprises: before the step of cyclizing the single-strand DNA fragment, phosphorylating the 5′ end and dephosphorylating the 3′ end of the DNA fragment. In some embodiments, the detection further comprises enriching the amplification product containing specific genome regions using an oligonucleotide probe before the sequencing step.

In some embodiments, the method provided in the present application can detect samples containing as little as ing, 2 ng, 3 ng, 4 ng, 5 ng, 6 ng, 7 ng, 8 ng, 9 ng, 10 ng, 15 ng, 20 ng, 25 ng, 30 ng, 45 ng, 50 ng, 55 ng, 60 ng, 65 ng, 70 ng, 75 ng, 80 ng, 85 ng, 90 ng, 95 ng, and 100 ng DNA. In some other embodiments, the method provided in the present application can detect samples containing 1-100 ng or over 100 ng DNA. In some preferred embodiments, the method provided in the present application can detect samples containing DNA within the scope of 20+5 ng. In some embodiments, the library conversion rate of DNA in the method provided in the present application is 10-90%, 10-80%, 10-70%, 10-60%, 10-50%, 10-40%, 10-30%, 10-20%, 20-90%, 20-80%, 20-70%, 20-60%, 20-50%, 20-40%, 20-30%, 30-90%, 30-80%, 30-70%, 30-60%, 30-50%, 30-40%, 40-90%, 40-80%, 40-70%, 40-60%, 40-50%, 50-90%, 50-80%, 50-79%, 50-60%, 60-90%, 60-80%, 60-70%, 70-90%, 70-80%, and 80-90%. In some other embodiments, the method provided in the present application can detect at least 100, 200, 300, 400, 500, 1000, 10⁴, 10⁵ methylated sites in 10 ng DNA. In yet some other embodiments, the coverage rate of bait/target DNA in the method provided in the present application is no less than 30%, 35%, 40%, 45%, 50%, 55%, 60%, 70%, 80%, 90%, 95%, 98%, or even higher.

### BRIEF DESCRIPTION OF THE DRAWINGS

The aforesaid and other features of the present application are better described through a combination of the drawings with the following specification and attached claims. It can be understood that these drawings only displays several embodiments of the present application, and thus should not be regarded as limitation on the scope of the present application. Through the adoption of the drawings, the present application shall be illustrated more explicitly and in more details.

FIG. 1: a schematic of the principle for treatment with sodium bisulfite;

FIG. 2: an embodiment of detecting the methylation of specific genome regions in a DNA fragment from a sample containing DNA;

FIG. 3: a schematic of the cyclization treatment of single-strand DNA;

FIG. 4: a schematic of inverse PCR amplification of a sequence containing methylated sites;

FIG. 5: a schematic of the rolling circle amplification of a sequence containing methylated sites;

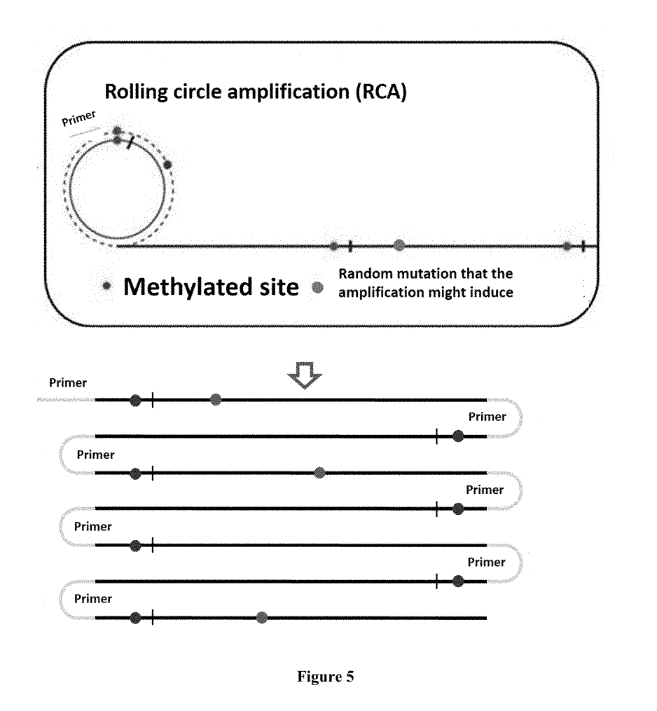

FIG. 6: comparison of the fragment size of the sequencing library prepared after treatment with sodium bisulfite with that of the sequencing library prepared without treatment with sodium bisulfite.

### DETAILED DESCRIPTION OF THE INVENTION

One aspect of the present invention provides a method of detecting the methylation of specific genome regions in a DNA fragment from a sample containing DNA.

The term “DNA” as used in the present invention is deoxyribonucleic acid, a long chain polymer biological macromolecule which forms genetic instructions. The subunit of DNA is nucleotide. Each nucleotide in DNA consists of a nitrogenous base, a five-carbon sugar (2-deoxyribose) and phosphate groups. Neighboring nucleotides are linked via diester bonds formed by deoxyribose and phosphoric acid, thereby forming a long chain framework. Generally there are four types of nitrogenous bases in DNA nucleotides, namely adenine (A), guanine (G), and cytosine (C), thymine (T). The bases on the two DNA long chains pair via hydrogen bonds, wherein adenine (A) pairs with thymine (T), and guanine (G) pairs with cytosine (C).

The term “sample containing DNA” as used in the present invention is any sample containing DNA fragments, including but not limited to cells, tissues, and body fluids, etc. In some embodiments, the sample containing DNA is a tissue, e.g., biopsy tissue or paraffin embedded tissue. In some embodiments, the sample containing DNA is a cell, e.g., bacteria (including virus) or animal or plant cell, etc. In some other embodiments, the sample containing DNA is a body fluid, e.g., blood, plasma, serum, saliva, amniocentesis fluid, pleural effusion, seroperitoneum, etc. In some specific embodiments, the sample containing DNA is blood, serum or plasma. In some specific embodiments, DNA contained in the sample is genomic DNA. In some specific embodiments, DNA contained in the sample is cell-free DNA (cfDNA). In some preferred embodiments, DNA contained in the sample is circulating tumor DNA (ctDNA).

“Cell-free DNA” refers to DNA free from cells found in circulatory system (e.g., blood), the source of which is generally believed to be genomic DNA released during apoptosis. Studies showed that the size of most cell-free DNA in human body is about 160 bp (see Fan et al., (2010) Analysis of the Size Distributions of Fetal and Maternal Cell-Free DNA by Paired-End Sequencing, Clin Chem 56:8 1279-86).

“Circulating tumor DNA” refers to the cell-free DNA originated from tumor cells. In human body, a tumor cell may release its genomic DNA into the blood due to causes such as apoptosis and immune responses. Since a normal cell may also release its genomic DNA into the blood, circulating tumor DNA usually constitutes only a very small part of Cell-free DNA.

The term “DNA methylation” as used in the present invention refers to a form of epigenetic modification. In eukaryotes, methylation of DNA only occurs to cytosine, specifically referring to a modification under catalysis of DNA methyltransferase (DNMTs), where a methyl group is transferred to cytosine and the cytosine is converted into 5-methylcytosine (mC). Studies showed that DNA methylation has significant influence on the function of DNA. In particular, DNA methylation may result in alteration in chromatin structure, DNA conformation, DNA stability and the way of interaction between DNA and proteins, thereby regulating gene expression. For example, methylation in the sequence of gene promoter usually inhibits the expression of the gene. It is found in some studies that DNA methylation is necessary for the normal development of organism. It is found in other studies that DNA methylation is related to genomic imprinting, X chromosome inactivation, and inactivation of repetitive elements. In particular, it is found in some studies that DNA methylation is relevant to the formation of tumors.

“Specific genome regions in a DNA fragment” as described in the present invention refers generally to all target regions for detection. In some preferred embodiments, the specific genome regions in a DNA fragment refer to a region rich in CpG, especially a CpG island region. In some other preferred embodiments, the specific genome regions in a DNA fragment refer to a CpG island region, the methylation of which is relevant to diseases (e.g., tumor, inflammation, birth defects, etc.).

In some embodiments, the method of detecting the methylation of specific genome regions in a DNA fragment from a sample containing DNA comprises the following steps: treating the DNA fragment containing specific genome regions with sodium bisulfite, where the treatment with sodium bisulfite converts unmethylated cytosine in the DNA fragment into uracil; denaturing the DNA fragment treated with sodium bisulfite to obtain a single-strand DNA fragment; attaching an adapter to one end or both ends of the single-strand DNA fragment; amplifying the adapter-attached single-strand DNA fragment using an index primer to prepare a DNA sequencing library containing the specific genome regions; and sequencing the DNA sequencing library to identify the sequence of the single-strand DNA fragment.

In some other embodiments, the method of detecting the methylation of specific genome regions in a DNA fragment from a sample containing DNA, comprising the following steps: treating the DNA fragment containing specific genome regions with sodium bisulfite, where the treatment with sodium bisulfate converts unmethylated cytosine in the DNA fragment into uracil; denaturing the DNA fragment treated with sodium bisulfite to obtain a single-strand DNA fragment; attaching an adapter to one end or both ends of the single-strand DNA fragment; cyclizing the adapter-attached single-strand DNA fragment; amplifying the adapter-attached single-strand DNA fragment using an index primer to prepare a DNA sequencing library containing the specific genome regions; and sequencing the DNA sequencing library to identify the sequence of the single-strand DNA fragment.

“Treatment with sodium bisulfite” as described in the present invention refers to treating the target DNA fragment using sodium bisulfite to convert unmethylated cytosine (C) in the target DNA fragment into uracil (U), while methylated cytosine remains unchanged. To be specific, at certain temperature and pH, cytosine in denatured DNA (single-strand DNA) is converted into cytosine-bisulfite derivative using sodium bisulfite, and then the cytosine-bisulfite derivative is deaminized by hydrolysis to obtain uracil-bisulfite derivative, and finally, uracil is obtained through the desulfonation of the uracil-bisulfite derivative under certain conditions; in this reaction, only unmethylated cytosine can undergo base change under treatment with bisulfite, while methylated cytosine remains unchanged. After the treatment with sodium bisulfite, due to the conversion of unmethylated cytosine into uracil, two originally complementary single-strand DNA may be no longer complementary. In some embodiments, the treatment of the target DNA fragment with sodium bisulfite may destruct the structure of the target DNA fragment, wherein the destruction refers to breaking the target DNA fragment treated with sodium bisulfite into smaller fragments.

In some embodiments, before treating with sodium bisulfite, the target DNA fragment is treated with a “type II restriction endonuclease”. “Type II restriction endonuclease” as described in the present invention refers to endonuclease which can identify and cleave specified sequences with 4-8 base pairs, wherein most identified sequences have palindrome structure; there are three cleavage patterns of type II restriction endonucleases: 1) the cleavage produces 5′ overhanging sticky ends, 2) the cleavage produces 3′ overhanging sticky ends, 3) the cleavage produces blunt ends. “Type II restriction endonuclease” as described in the present invention includes any type II restriction endonuclease known to a person of skill in the art, including but not limited to: ApaI, BamHI, BgIII, EcoRI, HindIII, HpaII, KpnI, MspI, NcoI, NdeI, NheI, NotI, SacI, Salt SmaI, SphI, XbaI, XhoI, XmaI. In some embodiments, the type II restriction endonuclease is selected from the group consisting of: HpaII, MspI, SmaI, XmaI, BamHI. In some embodiments, the type II restriction endonuclease is a methylation-sensitive restriction endonuclease. A “methylation-sensitive restriction endonuclease” is sensitive to the methylation state of DNA, the efficiency of which in cleaving methylated and unmethylated sequences is different, and thereby can be used to distinguish methylated sequences from unmethylated ones. In some embodiments, the type II restriction endonuclease is a methylation-sensitive restriction endonuclease, which is selected from HpaII, MspI, SmaI, XmaI, BamHI or any combinations thereof. In some embodiments, restriction endonucleases with different methylation sensitivities while capable of identifying identical DNA target sequences are used, for example, but not limited to, HpaII/MspI, SmaI/XmaI etc. In some embodiments, type II restriction endonuclease is used to break target DNA fragment into smaller random fragments. In some embodiments, DNA molecules without containing sequences with specific methylation modifications are background sequences that are not required to be ultimately sequenced, and the digestion of the regions without methylation in the target DNA fragment using type II restriction endonuclease can achieve the effect of reducing noises. In some other embodiments, DNA molecules with sequences with specific methylation modification are background sequences not required to be ultimately sequenced, and the digestion of the regions with methylation in the target DNA fragment using type II restriction endonuclease can achieve the effect of reducing noises. In some other embodiments, DNA molecules with specific methylation modification are DNA regions which need to be sequenced, and methylation state of such DNA regions can be learned from the identification of the cleavage sites through sequencing.

In some embodiments, if the size of the initial DNA fragment is >5 kb, the method of detecting the methylation of specific genome regions in a DNA fragment from a sample containing DNA further comprises cleaving the DNA fragment (including but not limited to physical breaking, cleavage using specific restriction endonuclease, etc.) before bisulfite treatment, to obtain DNA fragments with proper sizes for subsequent process, wherein the proper sizes for subsequent process are 0.01-5 kb, 0.1-5 kb, 0.1-1 kb, 1-2 kb, 2-3 kb, 3-4 kb, 4-5 kb, 0.2-0.4 kb, 0.5-1 kb, 0.1-0.5 kb, 0.01-0.5 kb, 0.01-0.4 kb, 0.01-0.3 kb, 0.01-0.25 kb, 0.02-0.25 kb, 0.05-0.3 kb or 0.05-0.25 kb. The cleaved DNA fragments contain the specific genome regions (target regions for detection). In some embodiments, if the size of the initial DNA fragment is >0.5 kb, it is required to cleave the DNA fragment before the treatment with bisulfite to obtain DNA fragments with sizes of 0.01-5 kb, 0.1-5 kb, 0.1-1 kb, 1-2 kb, 2-3 kb, 3-4 kb, 4-5 kb, 0.2-0.4 kb, 0.5-1 kb, 0.1-0.5 kb, 0.01-0.5 kb, 0.01-0.4 kb, 0.01-0.3 kb, 0.01-0.25 kb, 0.02-0.25 kb, 0.05-0.3 kb or 0.05-0.25 kb.

The method of “denaturing a DNA fragment to obtain a single-strand DNA fragment” as described in the present invention refers to any method known to a person of skill in the art, including but not limited to thermal denaturation (e.g., over 90° C.), alkali (e.g., NaOH) treatment, etc.

In some embodiments, the DNA fragments are cleaved before the treatment with sodium bisulfite to obtain DNA fragments with sizes of 0.01-5 kb, 0.1-5 kb, 0.1-1 kb, 1-2 kb, 2-3 kb, 3-4 kb, 4-5 kb, 0.2-0.4 kb, 0.5-1 kb, 0.1-0.5 kb, 0.01-0.5 kb, 0.01-0.4 kb, 0.01-0.3 kb, 0.01-0.25 kb, 0.02-0.25 kb, 0.05-0.3 kb or 0.05-0.25 kb, where the DNA fragments obtained from the cleaving contain the specific genome regions.

In some embodiments, the DNA contained in the sample is cell-free DNA. In some embodiments, the cell-free DNA contained in the sample is preferably circulating tumor DNA.

In some embodiments, an adapter is attached to one or both ends of the DNA fragment (single stranded or double stranded).

“Adapter” as described in the present invention refers to a specific DNA sequence attached to one or two ends of a DNA fragment (single stranded or double stranded) according to needs, the length of which is usually within 5-50 bp.

In some embodiments, where the DNA fragment is double stranded, the single-strand adapter can be designed to contain sequences or parts to ligate with an end of the DNA fragment (for example, a hybridization complementary region, or a random hybridization short sequence, e.g., poly-T), then through molecular hybridization of the adapter and the complementary strand of the target DNA fragment, and adding polymerase (e.g., reverse transcriptase) after the hybridization to extend the adapter, the adapter is ligated to the end of the target DNA fragment. In some other embodiments, where the DNA fragment is double stranded and the end of the DNA fragment is a sticky end, through designing the sequence of the adapter and using the sequence complementary to the adapter to complementarily anneal with it to form a structure with a sticky end, followed by ligating the complementary short sequence to the double strands of the target DNA using a ligase, and denaturing the DNA to form a single strand in the subsequent processes, thus achieving the purpose of attaching the adapter to the end of the DNA fragment. In some embodiments, an adapter that is bisulfite resistant is attached before the treatment with bisulfite. “Bisulfite resistant” as described in the present invention means that the adapter does not contain cytosine or does not contain unmethylated cytosine, so that when the DNA fragment is under the treatment with sodium bisulfite, the sequence of the adapter does not alter.

In some preferred embodiments, the double-strand DNA is denatured to obtain single-strand DNA fragments, followed by ligating the adapter sequence to one end (3′ end) or both ends of the single-strand DNA fragment using a single-strand DNA ligase. In some embodiments, the first adapter sequence (3′ adapter sequence) is ligated to the 3′ end of the single-strand DNA fragment using a single-strand DNA ligase, and then the single-strand one end adapter-attached DNA fragment is subject to N (N is an integer greater than or equal to 1) rounds of amplification using a linear amplification primer at least containing sequence partly complementary to the 3′ end adapter sequence, to obtain N times of the original copies of sequence complementary to the single-strand DNA fragment with an adapter sequence on the 3′ end. In some embodiments, a second adapter sequence (5′ adapter sequence) is ligated to the single-strand DNA fragment with adapter sequence attached to one end (amplified or not amplified) using a single-strand DNA ligase. In some other embodiments, where the DNA fragment is single stranded, the adapter sequence can be ligated to the end of the DNA fragment using a single strand ligase after phosphorylation and/or dephosphorylation of the DNA fragment and the adapter sequence. “Single-strand DNA ligase” as described in the present invention refers to any enzyme that can ligate two DNA known in the art, for example but not limited to T4 RNA Ligase, T4 DNA Ligase, Taq DNA Ligase, E. coli DNA Ligase, Ampligase (Epicentre).

The first adapter sequence ligated to the 3′ end of the single-strand DNA fragment and the second adapter sequence ligated to the 3′ end of the sequence complementary to the single-strand DNA fragment can be identical, different, complementary, partly complementary, containing partially identical sequence, and containing partially complementary sequence. In some embodiments, the adapter comprises a molecular tag. The term “molecular tag” as used in the present disclosure refers to a sequence used as a tag, which can be ligated to the 5′ end, the 3′ end or both ends of a DNA fragment. In DNA sequencing, especially in high throughout sequencing technology, a molecular tag is used to mark particular sequence. After amplification and sequencing, the count of the tag sequence can be the basis for determining the quantity of expression of the marked gene, or be used to trace the information of the amplified DNA molecules from the same original molecules and thereby correcting the random errors of DNA sequences during amplification and sequencing. A molecular tag can be a sequence of 4-20 bases, a random sequence (i.e., formed with randomly arranged A/T/C/G), and a fixed sequence. For example, 16 molecular tag sequences with 8 bases currently in use are as below: GACGTGAT, ACCACTGT, ACTTACGC, CTGTAGCT, GTAAGGAG, CACTTCGA, CATACCTG, TCGTGAGA, CGAGTGTA, CTATCTGC, TGGAACAC, AACTCACG, AATCCGAC, CAAGGAGT, GCATCCTA, CCTCTATC.

In some embodiments, the method provided in the present disclosure further comprises cyclizing the adapter-attached single-strand DNA fragment before the preparation steps of the library. “Method of cyclizing the single-strand DNA fragment” as described in the present invention refers to any method known to a person of skill in the art, including but not limited to cyclizing the single-strand DNA using highly efficient cyclizing agent. In some embodiments, the highly efficient cyclizing agent is a DNA ligase. In some embodiments, the DNA ligase is a single-strand DNA circligase (for example but not limited to CircLigase™ ssDNA Ligase, Epicentre Technologies Corporation), which is a thermally stable, ATP-dependent ligase that can catalyze the ligation between the 5′-phosphate and 3′-hydroxyl group of one single-strand DNA and thereby cyclizing the single-strand DNA. CircLigase™ ssDNA Ligase is different from T4 DNA Ligase and Ampligase® DNA Ligase. T4 DNA Ligase and Ampligase® DNA Ligase can only ligate the ends of DNA sequences that are adjacent and complementary to each other, while CircLigase™ ssDNA Ligase can ligate ends of a single-strand DNA without the presence of complementary sequences. Linear single-strand DNA with more than 15 bases including cDNA, can all be cyclized by CircLigase. Therefore, this enzyme plays an important role in ligating a linear single-strand DNA to form a cyclic single-strand DNA. Cyclic single-strand DNA molecules can be used as the substrate for rolling circle replication or rolling circle transcription studies.

In some embodiments, the 5′ end of the DNA fragment is phosphorylated and the 3′ end dephosphorylated before cyclizing the adapter-attached single-strand DNA fragment. The phosphorylation of the 5′ end and the dephosphorylation of the 3′ end can be conducted using any method known to a person of skill in the art, including but not limited to catalyzing using a T4 polynucleotide kinase (T4 PNK). T4 PNK is a polynucleotide 5′ hydroxyl kinase, which can catalyze the transferring of the γ phosphate group of ATP to the 5′ hydroxyl of single-strand or double-strand DNA, RNA, oligonucleotides or mononucleotides with 3′ phosphate group. Other NTPs can generate the same reaction: 5′-OH+NTP→5′-P+NDP. T4 PNK also has 3′ phosphatase activity, and can catalyze the dephosphorylation of 3′ phosphorylated polynucleotides: 3′-P→3′-OH+Pi (the optimum pH is about 5.9). The kinase activity of T4 PNK is somewhere near the C-end, and the phosphatase activity is near the N-end, and can therefore be used in phosphorylating the 5′ end and/or removing the 3′ end phosphate group of oligonucleotides, DNA or RNA, to ensure the success of the subsequent ligating reaction.

In the present disclosure, the “index primer” used to prepare a DNA sequencing library may refer to a single primer or paired primers, containing respectively part of or the entire sequence identical or complementary to the first adapter sequence and the second adapter sequence.

“DNA sequencing library” as described in the present disclosure refers to a collection of DNA segments, in an abundance that can be sequenced, wherein one end or both ends of each segment in the collection of DNA segments contains a specific sequence partly or completely complementary to the primer used in sequencing, and thereby can be directly used in the subsequent DNA sequencing.

In some embodiments, the process of preparing the cyclized adapter-attached single-strand DNA fragment into a DNA sequencing library comprises: after the cyclization process, amplifying the cyclized adapter-attached single-strand DNA fragment, and obtaining the DNA sequencing library comprising the amplification product of the DNA containing the specific genome regions. In some embodiments, the amplification steps uses inverse PCR amplification. In some embodiments, the amplification steps uses rolling circle amplification.

“Inverse PCR” as described in the present invention refers to the process that, where there is a known sequence, through designing a primer based on the known sequence, and synthesizing a DNA on the exterior of the primer, the flanking DNA of the known sequence is amplified. In some embodiments of the present invention, the known sequence in the inverse PCR is a known sequence in the specific genome regions of the DNA fragment. In some other embodiments of the present invention, the known sequence in the inverse PCR may be part of or the entire sequence of the adapters attached to the ends of the DNA fragment as aforesaid.

“Rolling circle amplification” as described in the present invention means that a primer is extended under DNA polymerase after combination with a cyclic DNA, generating a linear DNA single strand with a large number of repetitive sequences (completely complementary to the cyclic DNA). Similarly, in some embodiments of the present invention, the primer in the rolling circle amplification may be from a known sequence in the specific genome regions of the DNA fragment. Or, in some other embodiments of the present invention, the primer in the rolling circle amplification may contain sequence partly or completely complementary to that of the adapters attached to the ends of the DNA fragment.

In some embodiments, amplification after cyclizing the DNA can reduce the dependence on the length of the template DNA fragment during amplification, which is beneficial for amplifying segmented DNA (e.g., cell-free DNA in plasma). For example, traditional double-primer forward PCR amplification technology requires that the template DNA to be amplified contains the sequences of both primers at the same time, but when the length of one template DNA is too short, it cannot contain the sequences of both primers at the same time, under which circumstance such DNA template cannot be amplified. However, in inverse PCR, since the two primers can be very close to each other, it is more beneficial for amplifying segmented DNA (e.g., free DNA in plasma). Therefore, a higher sensitivity for detecting segmented DNA can be achieved through inverse PCR after cyclization.

In some embodiments, the sequencing steps determines the sequence of the amplification product using high throughout DNA sequencing technology.

When the sodium bisulfite treated DNA is sequenced, or after amplification (e.g., inverse PCR amplification or rolling circle amplification) of the aforesaid sodium bisulfite treated DNA, uracil (U) converted from unmethylated cytosine (C) will transform into thymine (T). By sequencing the bisulfite treated DNA fragment and comparing the result with the sequence of the DNA fragment that is not bisulfite treated (known sequence or the result obtained by sequencing the DNA fragment that is not bisulfite treated), methylation of specific genome regions in the DNA fragment can be determined. Specifically, regions where the conversion from C to T is found through comparison of the sequencing results are unmethylated regions.

In some embodiments, the steps of preparing the single-strand DNA fragment into a DNA sequencing library in the method further comprise: enriching specific amplification product (e.g., amplification product containing specific sequences or amplification product with specific molecular tag) using an oligonucleotide probe, after the amplification process. The steps of “enriching DNA amplification product containing the specific genome regions using an oligonucleotide probe” as described in the present invention can be completed through any method known to a person of skill in the art, for example but not limited to magnetic beads enrichment, tag pull-down, etc.

Another aspect of the present invention provides a kit for detecting the methylation of specific genome regions in a DNA fragment from a sample containing DNA. In some embodiments, the kit comprises: sodium bisulfite, which can convert unmethylated cytosine in the DNA fragment into uracil; a single-strand DNA ligating agent, which can ligate a single-strand DNA fragment with an adapter sequence; and a sequencing agent. In some embodiments, the single-strand DNA ligating agent comprises an adapter sequence DNA fragment, a single-strand DNA ligase, a reacting solution. In some embodiments, the single-strand DNA ligase is a T4 RNA Ligase. In some embodiments, the kit further comprises a linear amplification agent, the linear amplification agent comprising a linear amplification primer, which comprises part of or the entire sequence complementary to an adapter attached to one end of the single-strand DNA fragment. In some embodiments, the linear amplification primer further comprises a DNA polymerase, a reaction solution and dNTPs. In some embodiments, the adapter sequence DNA fragment comprises one or more selected from the groups consisting of: a sequence partly or completely complementary to the sequence of the restriction endonuclease site, molecular tag sequence, and a sequence partly or completely complementary to the linear amplification primer.

In some embodiments, the kit further comprises a library preparing agent, which can be used to amplify the single-strand DNA containing specific genome regions, the library preparing agent comprising an index primer. In some embodiments, the index primer contains respectively the sequences partly or completely identical or complementary to the sequence of the first/second adapter. In some embodiments, the index primer also contains the sequence partly or completely complementary to the capturing sequence of the sequencing platform used in subsequent sequencing steps. In some embodiments, the library preparing agent further comprises an amplification agent. In some embodiments, the amplification agent comprises a DNA polymerase, a reaction solution and dNTPs.

In some embodiments, the kit further comprises a cyclization agent, which can cyclize the single-strand DNA fragment. In some embodiments, the cyclization agent is a highly efficient cyclization agent. In some embodiments, the highly efficient cyclization agent is a single-strand DNA circligase. In some embodiments, the single-strand DNA circligase is CircLigase. In some embodiments, the cyclization agent further comprises T4 PNK, which can be used in phosphorylation of the 5′ end and dephosphorylation of the 3′ end of the DNA fragment. In some embodiments, the library preparing agent comprises an agent used in inverse PCR amplification or an agent used in rolling circle amplification.

In some embodiments, the kit further comprises a cleaving agent, which can be used in cleaving the DNA fragment before the treatment with sodium bisulfite to obtain DNA fragments with proper sizes for subsequent process, wherein the proper sizes for subsequent process are 0.1-5 kb, 0.1-1 kb, 1-2 kb, 2-3 kb, 3-4 kb, 4-5 kb, 0.1-0.5 kb, 0.5-1 kb or 0.2-0.4 kb. The cleaved DNA fragments contain the specific genome regions (target regions for detection). In some embodiments, the cleaving agent is a saline solution. In some embodiments, the cleaving agent is a TE solution (The coordinating with the use of a DNA physical breaking device not included in the kit is required). In some other embodiments, the cleaving agent is an endonuclease (or an endonuclease together with its reaction solution).

Yet another aspect of the present invention provides use of a single-strand DNA ligating agent in preparation of a kit for detecting the methylation of specific genome regions in a DNA fragment from a sample containing DNA, wherein the detection comprises the following steps: treating the DNA fragment containing specific genome regions with sodium bisulfite, where the treatment with sodium bisulfite is able to convert unmethylated cytosine in the DNA fragment into uracil and to obtain a single-strand DNA fragment; attaching an adapter to one end or both ends of the single-strand DNA fragment using the single-strand DNA ligating agent; amplifying the adapter-attached single-strand DNA fragment using an index primer, to prepare a DNA sequencing library containing the specific genome regions; sequencing the DNA sequencing library. In some embodiments, an adapter is attached to one end of the single-strand DNA fragment via a single-strand DNA ligase. In some embodiments, the detection further comprises: linear amplification of the one end adapter-attached single-strand DNA fragment. In some embodiments, the detection further comprises: attaching an adapter to the other end of the amplification product of the one end adapter-attached single-strand DNA fragment using a single-strand DNA ligase. In some embodiments, the detection further comprises cyclizing the adapter-attached single-strand DNA fragment using a highly efficient cyclizing agent, and amplifying the cyclized single-strand DNA fragment using an index primer after the cyclization step, wherein the sequencing step is to determine the sequence of the amplification product.

In some embodiments, the detection further comprises: before cyclizing the single-strand DNA fragment, phosphorylating the 5′ end and dephosphorylating the 3′ end of the DNA fragment.

In some embodiments, the detection further comprises enriching the amplification product of the DNA fragment containing specific genome regions using an oligonucleotide probe before the sequencing step.

### Examples

The present invention is further described below through some non-restrictive examples. It needs to be noted that these examples are only used for further illustrating the technical features of the present invention, which are not intended to, nor can be interpreted as limiting of the present invention. These examples do not include elaboration of the traditional methods known to a person of skill in the art (the extraction, purification, etc., of DNA in different types of samples).

### Example 1: The IRIS Assay

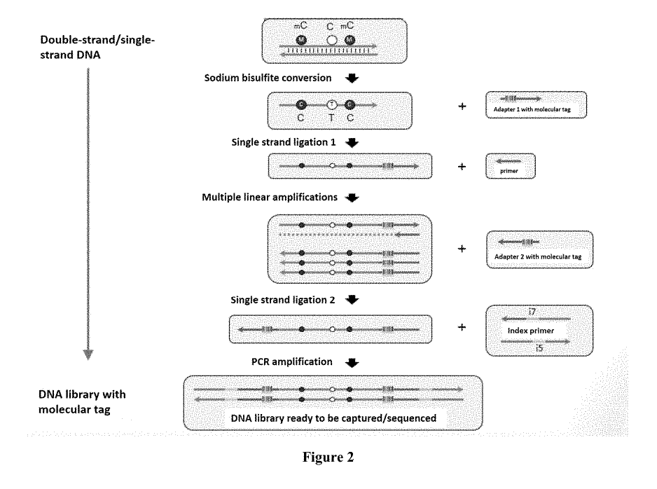

The following provides a specific embodiment of the method for detecting the methylation of specific genome regions in a DNA fragment from a sample containing DNA, as described in the present invention (the IRIS assay). This specific embodiment can be understood with reference to FIG. 2 and the following specific description.

### Treatment of the Sample

Where the target DNA was genomic DNA in a cell or a tissue, the genomic DNA contained in the sample was extracted or enriched using a DNA extraction kit or using a DNA enrichment tool (DNA absorbing column, magnetic beads, etc.), etc., and the concentration of DNA was measured using Nanodrop and recorded. 2 μg DNA was taken and diluted to 200 μl with double distilled water, and the aforesaid DNA was broken into fragments of about 200-800 bp through digestion, ultrasonic breaking, repetitive freeze-thawing, and the like, with the choice of a DNaseI enzyme or restrictive endonuclease according to practical needs (optional step). Usually ultrasonic breaking is used with the operation parameters of: aptitude 40, power 50 w, duration 15 sec, interval 5 sec, times 15, under low temperature of 0-4° C. (the ultrasonic parameters can be tried and adjusted according to practical conditions). The size of the DNA fragment after the treatment was confirmed to be within the required scope, using gel electrophoresis.

Where the target DNA is cfDNA, the short sequence (115 bp) and the long sequence (247 bp) of the ALU gene of high abundance in human genomic DNA from the enriched cfDNA can be detected using qRT PCR, wherein the 115 bp ALU amplicon (ALU115) and the 247 bp ALU amplicon (ALU247) corresponds respectively to the concentration of total DNA (cfDNA and genomic DNA) and the genomic DNA, and thereby the content of cfDNA in the sample can be calculated. Moreover, the concentration ratio of ALU247/ALU115 represents the completeness of the DNA in the sample. Optionally, the enriched cfDNA can be treated using MspI or other similar type II restrictive endonucleases, to be broken into fragments of 20-400 bp.

Treatment with Sodium Bisulfite

An appropriate amount of the aforesaid DNA was added into a 1.5 ml EP tube, and 5.5 μl freshly prepared 3M NaOH solution was added in, the EP tube was placed in a water bath of 42° C. to incubate for 30 min (during which DNA denaturation occurred, generating single-strand DNA). During water bathing, 10 mM hydroquinone and 3.6 mM sodium bisulfite solutions were prepared, wherein the steps for preparing the 3.6M sodium bisulfite solution were as below: 1.88 g sodium bisulfite was dissolved using double distilled water, and the solution was titrated with 3M NaOH until the pH is 5.0, then filled up the solution to 5 ml. After incubating for 30 min in 42° C. water bath, 30 μl aforesaid 10 mM hydroquinone was charged into an EP tube (the color of the solution turned into light yellow), and then 520 μl aforesaid 3.6M sodium bisulfite solution was added into the EP tube. The EP tube was wrapped with aluminum foil, and gently turned upside down to mix the solution, and then 200 μl paraffin sealing liquid was added into the EP tube to prevent water evaporation. The EP tube was further placed in a water bath of 50° C. to incubate away from light for 16 hrs (to complete the conversion from cytosine-bisulfite derivative to uracil-bisulfite derivative). Then the DNA containing uracil-bisulfite derivative was absorbed on a purification column (using commercially available DNA purification column, etc.), and 50 μl DNA eluent was obtained after purification and elution. The tube was kept at room temparture for 15 min after 5.5 μl freshly prepared 3M NaOH was added; then 33 μl 10M ammonium acetate was added to neutralize NaOH, adjusting the pH of the solution to about 7.0, and 4 μl 10 mg/ml glycogen was added as precipitation indicator (mixing glycogen with ethanol generates precipitation, which helps to identify the recovery product after precipitating with ethanol and centrifuging); finally 270 μl icy (0-4° C.) anhydrous ethanol was added into the solution, and the EP tube was placed in an environment of −20° C. for 2-6 hrs (or overnight) to precipitate DNA (corresponding to the process of desulfonation). The precipitated DNA may be dried after being washed with 70% ethanol repeatedly, and after re-dissolving in double distilled water, a DNA solution modified through sodium bisulfite treatment was obtained.

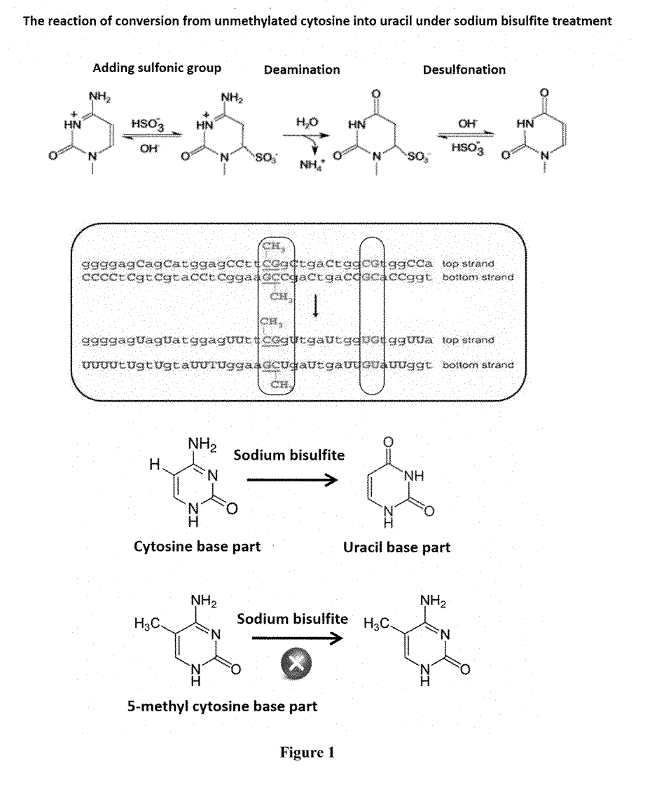

In this reaction, only unmethylated cytosine can undergo base change under treatment with bisulfite, and methylated cytosine remains unchanged (see FIG. 1).

It should be understood that, the aforesaid experiment operation of treatment with sodium bisulfite is exemplary only. A person of skill in the art can complete this step using any commercially available sodium bisulfite treatment kit, wherein the agent, reaction condition, reaction time, etc. used in this step are different but the basic principles are substantially the same.

Since the sodium bisulfite treatment includes alkali treatment, which thereby denatures the initial DNA into a single-strand DNA, and since the DNA fragment mostly remains the denatured form during the treatment with sodium bisulfite, a DNA fragment treated with sodium bisulfite is usually a single-strand DNA fragment. Optionally, denaturation can be further included after treatment with sodium bisulfite to ensure that all DNA fragments are single-strand DNA fragments.

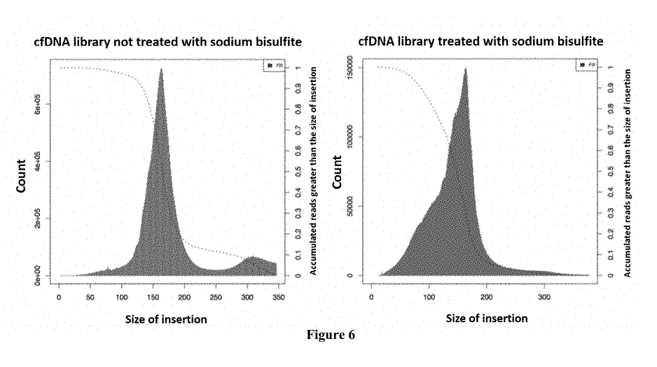

In addition, treatment with sodium bisulfite sometimes leads to breakage of a DNA fragment, and therefore, preferably, an adapter is added to one end or both ends of the DNA fragment after treatment with sodium bisulfite. As FIG. 6 shows, compared with the sequencing library prepared without treatment with sodium bisulfite, the sequencing library produced after treatment with sodium bisulfite followed by amplification has significantly reduced DNA fragments that are >250 bp, and significantly increased DNA fragments with sizes of 50-100 bp, which shows that treatment with sodium bisulfite destructs DNA fragments and breaks them into smaller fragments.

Adapters of the DNA Fragment After treatment with bisulfite, a specific sequence can be attached to one end or both ends of the DNA fragment (single stranded or double stranded) according to practical needs. The specific sequence attached to the ends of the DNA fragment is known as first adapter and/or second adapter respectively.

In one embodiment, the first adapter sequence (3′ adapter sequence) and the second adapter sequence (5′ adapter sequence) were ligated to the ends of a single-strand DNA fragment, using a single-strand DNA ligase such as T4 RNA ligase. The reaction solution was prepared according to the following formula: 2 μl 10×T4 RNA Ligase ligation reaction solution, 1 μl 10 mM ATP, 50-100 pmol single-strand DNA fragments, 50-100 pmol adapter sequence (substantially equal to the amount of single-strand DNA fragments), 1 μl (10 u) T4 RNA Ligase, filling up the reaction system to 20 μl by adding double distilled water. The reaction system was incubated at 4° C. for 10-18 hours.

In another embodiment, first adapter sequence (3′ adapter sequence) was ligated to the 3′ end of a single-strand DNA fragment using T4 RNA ligase, and then the one end adapter-attached single-strand DNA fragment was subject to N=1-30 rounds of amplification using a linear amplification primer at least containing sequence partly complementary to the 3′ end adapter sequence, obtaining N times of the original copies of sequence complementary to the single-strand DNA fragment with an adapter sequence on the 3′ end.

Linear amplification can be used in amplifying the sequence of an unknown segment adjacent to a known DNA segment, which differs from ordinary PCR in that it uses only one primer in the linear amplification, and the advantage lies in the smaller discrepancy in amplification efficiencies for different amplification fragments, and better uniformity of different amplification products. The reaction conditions used in linear amplification are similar to that of the classic polymerase chain reaction, for example, using Taq polymerase, after 94° C. denaturation of DNA for 30 seconds, repeating 1-30 cycles of 58° C. primer annealing for 30 seconds and 70° C. extension for 3 minutes.

Subsequently, a second adapter sequence (5′ adapter sequence) was ligated to the 3′ end of the amplified sequence complementary to the single-strand DNA fragment with adapter sequence attached to the 3′ end using a single-strand DNA ligase such as T4 RNA Ligase in the method above, obtaining a single DNA fragment with adapters or sequences complementary to the adapters attached to both ends.

The first adapter sequence and the second adapter sequence can be identical, different, complementary, partly complementary, containing partially identical sequence, and containing partially complementary sequence. The adapter may contain a sequence to be used as a tag as a molecular tag. For another example, the adapter may contain a sequence complementary to part of or the entire region of the index primer used in the subsequent preparation of library.

In another embodiment, the first adapter may optionally contain a molecular tag or a specific sequence on its 3′ end or its 5′ end. The aforesaid molecular tag or specific sequence can be used in subsequent amplification, for specific amplification of the target DNA. Or aforesaid molecular tag or specific sequence can be used in subsequent sequencing, for specific enrichment of the molecules containing the specific sequence. Or in subsequent sequencing, the count of the tag sequence can be the basis for determining the quantity of expression of the tagged gene, or be used to trace the information of the amplified DNA molecules from the same original molecules and thereby correcting the random errors of DNA sequences during amplification and sequencing.

In some specific embodiments, the difference between the method provided in the present application and the prior art (for example but not limited to, the method of Illumina or the method of Swift Biosciences) lies in that the method of the present application includes: i) the attachment of the adapter sequence after the treatment with sodium bisulfite; ii) multiple rounds of linear amplification of the primer with 3′ end adapter after the first single strand ligation ligating the 3′ end adapter to the single-strand DNA converted by sodium bisulfite; iii) using the method of single strand ligation again after amplification, to ligate with the 5′ end sequencing adapter; iv) there are n (4<=n<=20) bases on the adapters in both single strand ligations as the molecular tag of UMI (Unique Molecular Identifiers), which are used to mark the original DNA molecules, to reduce background noises in later analysis, and to count the original molecules, etc.

DNA Sequencing

The DNA library is sequenced using the method of high throughout DNA sequencing. The target DNA to be sequenced can be enriched using an oligonucleotide probe before sequencing.

The sequencing for DNA methylation may be substantially through sequencing the bisulfite treated DNA fragment, or the amplified bisulfite treated DNA fragment, and comparing the sequencing result with the sequence of the DNA fragment without bisulfite treatment (known sequence or the sequencing result from sequencing the DNA fragment without bisulfite treatment), and then the regions where the conversion from C to T is found in the comparison of the sequencing results are prospective methylated regions. According to the frequency each prospective methylated region appears in the multiple copies, whether methylation actually exists on that region is evaluated.

### Example 2: Optional Steps of the IRIS Assay

Optionally, the IRIS assay may further include cyclizing the adapter-attached single-strand DNA fragment, and amplifying the cyclized single-strand DNA fragment to prepare the sequencing library.

DNA Cyclization

Optionally, the aforesaid bisulfite treated DNA fragment can be phosphorylated on the 5′ end and dephosphorylated on the 3′ end (see the content inside the dashed box of FIG. 2). Specifically, the DNA fragment can be treated using T4 PNK and a reaction solution prepared according to corresponding instruction. For example, adding 5-50 pmol single-strand DNA template, 5 μl 10×T4 PNK reaction solution, 1 μl 1 mM ATP and 10U T4 PNK enzyme, in 50 μl reaction system, and after filling up to 50 μl, placing the reaction system in 37° C. incubation for 30 minutes, and finally inactivating T4 PNK through 80° C. incubation for 10 min.

The bisulfite treated DNA fragment (with or without phosphorylation of the 5′ end and dephosphorylation of the 3′ end) was placed in 95° C. incubation for 2 min, denaturing the aforesaid DNA fragment into single-strand DNA fragment. A typical cyclization system is as shown in the following table:

TABLE 1
A typical cyclization system

| | Stock | | Final | | | |
|---|---|---|---|---|---|---|
| | concentra- | | concentra- | | Per | |
| Reagent | tion | Unit | tion | Unit | 40 uL | |
| T4 PNK treated | — | — | — | — | 20 | μL |
| template DNA | | | | | | |
| 10X reaction | 10 | X | 1 | X | 4 | μL |
| solution | | | | | | |
| MnCl₂ | 50 | mM | 5 | mM | 4 | μL |
| ATP | 100 | μM | 10 | μM | 4 | μL |
| CircLigase | 5 | X | 1 | X | 8 | μL |
| H₂O | — | — | — | — | — | μL |

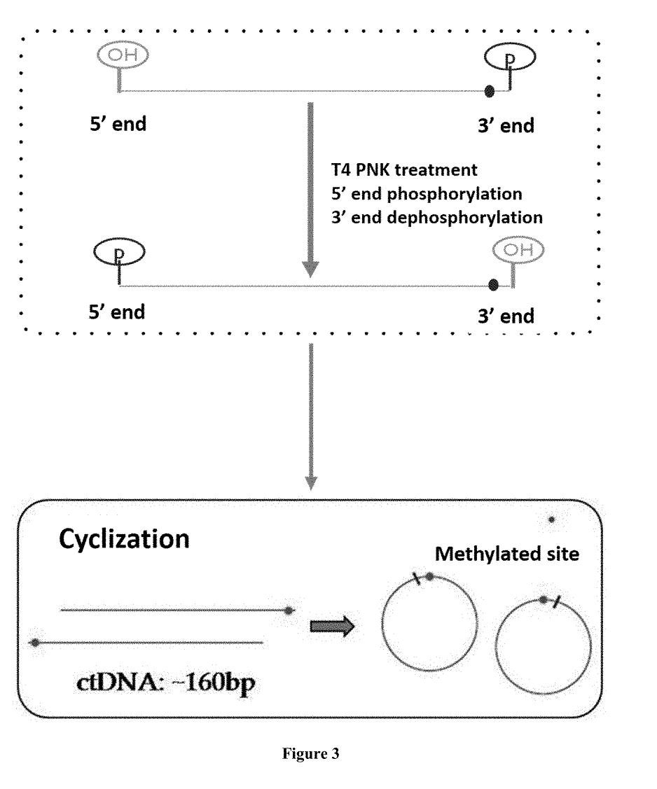

The reaction solution can be prepared, for example, according to the following formula: 10 pmol single-strand DNA template, 2 μl CircLigase10X reaction solution, 1 μl mM ATP, 1 μl 50 mM MnCl₂, 1 μl CircLigase™ ssDNA Ligase (100U), filled up to 20 μl, and then placing the reaction solution prepared above in 60° C. incubation for 1 hour, and finally placing the reaction solution in 80° C. incubation for 10 min to inactivate CircLigase™ ssDNA Ligase (see the content inside the solid box of FIG. 3).

Amplification of Cyclized DNA

The aforesaid cyclized DNA can directly be used in the subsequent sequencing, or where such cyclized DNA is low in abundance, it can be first amplified then the amplification product can be used in the subsequent sequencing steps.

Any method known to a person of skill in the art can be used for the amplification of cyclized DNA. The common ones include inverse amplification and rolling circle amplification.

Inverse PCR Amplification

Inverse PCR amplification can be used for studying the sequence of the unknown segment adjacent to the known DNA segment, as well as amplifying cyclic DNA fragments, wherein although the primers used are complementary to the two adjacent sequences within the known DNA segment (see primer pairs in FIG. 3: primer 1 and primer 2, primer 3 and primer 4; i.e., the 5′ ends of the two primers in every primer pair is adjacent), the 3′ ends of the two primers are mutually inverse. The linear amplification product of cyclic DNA is obtained through upstream and downstream extension of inverse PCR amplification primers (see FIG. 3 for details).

The reaction conditions used in inverse PCR amplification are similar to that of the classic polymerase chain reaction, for example, using Taq polymerase, after 94° C. denaturation of DNA for 30 seconds, repeating 30 cycles of 58° C. primer annealing for 30 seconds and 70° C. extension for 3 minutes.

Rolling Circle Amplification

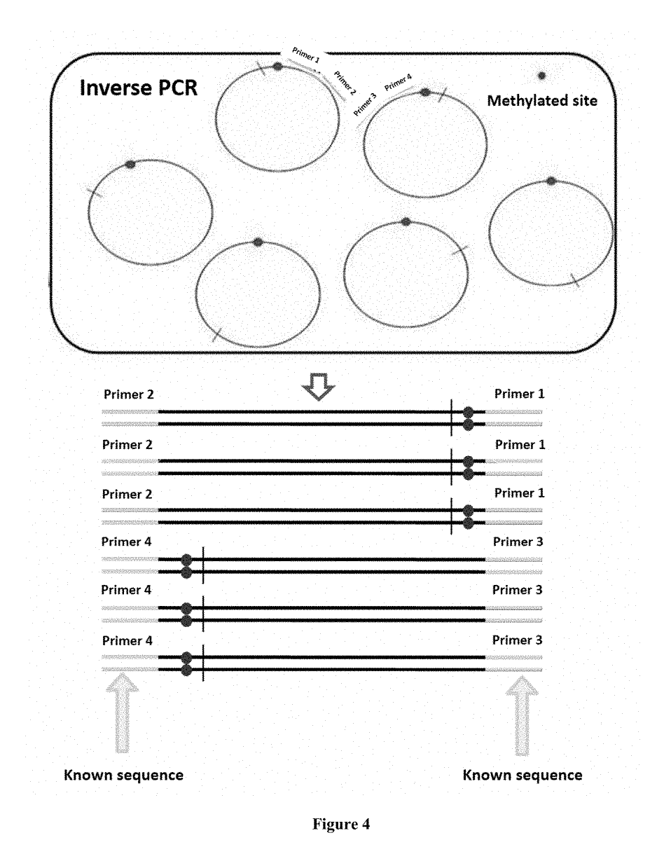

Cyclized DNA can be amplified using primers designed for the part of known sequence of the cyclized DNA or for part of the sequence of the adapter attached to one end or both ends of the cyclized DNA before cyclization, to generate multiple copies of sequence complementary to the linearly ligated cyclized DNA (see FIG. 4). The reaction conditions used in rolling circle amplification are also similar to that of the classic polymerase chain reaction, which will not be repeated herein.

### Example 3: Comparison Data Between the IRIS Assay and the SWIFT Assay

The IRIS assay was compared with the SWIFT® Accel-NGS-Methyl-Seq™ assay from Swift Biosciences, Inc. In this experiment, human cfDNA was used as the test object to compare the sensitivity, library forming efficiency, accuracy, mapping coverage, etc. of the two assays. The concentration of the cfDNA collected after enrichment was tested according to the method stated above, wherein ing, 3 ng, 5 ng, and 10 ng cfDNA was taken respectively for testing, and in each test, two samples were taken for parallel experiments. An IRIS library was prepared according to the method stated above, and meanwhile, a SWIFT library was constructed according to the instructions from the manufacturer (Cat# DL-ILMMS-12/48). The number of PCR cycles during linear amplification and/or preparation of the libraries was adjusted according to the input DNA amount, and the concentration of DNA in the libraries were tested using Qubit dsDNA HS kit. Next, a probe panel was used to enrich the constructed sequencing libraries respectively. 300-500 ng DNA library was taken, and NextSeq500 was used to sequence the enriched library, with the sequencing length read of 2×150 bp, and afterwards, sequencing reads were aligned to the sequence of human genome hg19 using bismark software. Library quality was evaluated based on the criteria listed in table 2.

All tested samples were analyzed with similar number of total sequencing reads (>10,000,000) with about 65%˜70% mapping rate. As FIG. 2 shows, in the SWIFT test, only samples with 10 ng DNA initial amount met the quality control requirements. Libraries prepared from samples with less DNA initial amount (5 ng, 3 ng or 1 ng) cannot achieve sufficient mean bait/target coverage and uniformity (i.e., the prepared libraries cannot achieve sufficient diversity), despite of their high mean bait coverage and on-target reads before deduplication. On the contrary, all tested IRIS libraries (prepared from samples with DNA initial amount of 10 ng, 5 ng, 3 ng and 1 ng) met the quality control requirements and were suitable for subsequent analysis, and even the IRIS libraries with ing DNA initial amount achieved better performance than the SWIFT libraries with 10 ng DNA initial amount. Library conversion rate was estimated respectively and compared between the IRIS and SWIFT assays according to the data resulted from the test. The estimated library conversion rate is the ratio of the estimated molecule number in a library divided by the theoretical molecule number corresponding to the input DNA amount. The estimated molecule number in a library is calculated through sequencing depth and detected sequencing diversity (detecting molecule number) based on the Poisson Distribution. Conversion rates for each cfDNA sample were calculated and summarized in table 3, the result of which shows that the IRIS assay can provide a conversion rate at least 5-fold greater than the SWIFT assay.

TABLE 2
Data results comparison of the IRIS assay and the SWIFT assay

| | | Input | | Total PF | On- | Pre-dedup | Deduped | Deduped | % target base | |
|---|---|---|---|---|---|---|---|---|---|---|
| | | cfDNA | Mapping | reads | target | mean bait | mean bait | mean target | coverage > | Uniformity |
| Sample | Assay | (ng) | % | (M) | % | coverage | coverage | coverage | 30X | (0.2X mean) |
| 1ng_IRM_Rep1 | IRIS | 1 | 65% | 11 | 36% | 670 | 155 | 92 | 80% | 90% |
| 1ng_IRM_Rep2 | IRIS | 1 | 65% | 10 | 37% | 638 | 151 | 90 | 80% | 91% |
| 3ng_IRM_Rep1 | IRIS | 3 | 68% | 11 | 35% | 643 | 268 | 161 | 91% | 91% |
| 3ng_IRM_Rep2 | IRIS | 3 | 67% | 13 | 36% | 762 | 288 | 172 | 92% | 91% |
| 5ng_IRM_Rep1 | IRIS | 5 | 69% | 11 | 39% | 738 | 350 | 211 | 95% | 92% |
| 5ng_IRM_Rep2 | IRIS | 5 | 67% | 10 | 39% | 626 | 318 | 191 | 93% | 91% |
| 10ng_IRM_Rep1 | IRIS | 10 | 70% | 12 | 45% | 881 | 481 | 290 | 97% | 92% |
| 10ng_IRM_Rep2 | IRIS | 10 | 70% | 11 | 44% | 815 | 447 | 270 | 97% | 91% |
| 1ng_SWT_Rep1 | SWIFT | 1 | 66% | 11 | 63% | 1427 | 20 | 12 | 8% | 82% |
| 1ng_SWT_Rep2 | SWIFT | 1 | 66% | 13 | 63% | 1704 | 21 | 12 | 9% | 83% |
| 3ng_SWT_Rep1 | SWIFT | 3 | 68% | 12 | 79% | 2083 | 48 | 29 | 40% | 85% |
| 3ng_SWT_Rep2 | SWIFT | 3 | 68% | 12 | 79% | 1988 | 41 | 25 | 34% | 84% |
| 5ng_SWT_Rep1 | SWIFT | 5 | 68% | 12 | 59% | 1542 | 65 | 40 | 52% | 85% |
| 5ng_SWT_Rep2 | SWIFT | 5 | 67% | 12 | 59% | 1488 | 64 | 39 | 51% | 85% |
| 10ng_SWT_Rep1 | SWIFT | 10 | 69% | 12 | 64% | 1608 | 113 | 69 | 68% | 85% |
| 10ng_SWT_Rep2 | SWIFT | 10 | 68% | 13 | 64% | 1763 | 107 | 65 | 67% | 84% |
| Quality control standard | | | >40% | >10 | >30% | >500 | >60 | >30 | >50% | >85% |

It should be understood that, although the means for performing some specific steps of the method disclosed in the present invention are described in details in the above examples, such description is only exemplary rather than limiting the present invention. In fact, based on the examples of the present invention, a person of ordinary skill in the art can understand and perform other variations of the disclosed embodiments, through studying the specification, the disclosed content and drawings, and the attached claims. The singular forms “a,” “an,” and “the” used herein intend to also include the plural forms, unless it is clearly indicated in the context otherwise. The terms “comprise”, “comprising”, “includes”, and/or “including as used herein specify the presence of stated features, integers, steps, operations, elements and/or components, but do not preclude the presence or addition of one or more other features, integers, steps, operations, elements, components and/or groups thereof. The values of volume, concentration, number, etc. disclosed herein should not be contemplated to be only limited to the exact value as recorded. In fact, unless otherwise indicated, each such value intends to express the recorded value as well as the functionally equivalent scope around the value. For example, a volume disclosed as “20 μL” intends to indicate “about 20 μL”. The “about”, “substantially”, “generally”, “approximately”, etc. as stated herein intend to indicate the value or scope ±5% of the described value or scope. Meanwhile, although the present invention has illustrated and described some specific embodiments, a person of skill in the art can clearly know that many other alterations and modifications can be made without deviating from the subject and scope of the present invention, and therefore the attached claims intend to cover all these alterations and modifications within the subject and scope of the present invention.

Unless explicitly excluded or otherwise limited, all contents of any document cited herein, including any cross-referenced or relevant patent or patent application, are incorporated herein by reference in its entirety. The citation herein to any document does not mean agreeing that the document itself or its combination with other reference document, teaching, suggestion, or any disclosure of such kind of inventions is the prior art of the invention disclosed or seeking to be protected herein. Furthermore, when the meaning or definition of any terminology herein contradicts with the meaning or definition of the same terminology in a cited document, the meaning or definition of the terminology as defined herein prevails.

What is claimed:

1. A method of detecting methylation within specific genome regions in a DNA fragment from a sample containing DNA, comprising steps of:
treating the DNA fragment containing the specific genome regions with sodium bisulfite, wherein the treatment with sodium bisulfite converts unmethylated cytosine in the DNA fragment into uracil, and obtaining a single-strand DNA fragment;
attaching an adapter to one end or both ends of the single-strand DNA fragment;
preparing a DNA sequencing library containing the specific genome regions by amplifying the adapter-attached single-strand DNA fragment with an index primer; and
sequencing the DNA sequencing library to determine sequence of the single-strand DNA fragment.

2. The method according to claim 1, further comprising: denaturing the DNA fragment treated with sodium bisulfite.

3. The method according to claim 1, wherein the adapter is attached to both ends of the single-strand DNA fragment via a single-strand DNA ligase.

4. The method according to claim 1, wherein the step of attaching the adapter comprises
attaching a first adapter to 3′ end of the single-strand DNA fragment using a single-strand DNA ligase,
linearly amplifying the single-strand DNA fragment with the first adapter attached on the 3′ end to obtain an amplification product, and
attaching a second adapter to 3′ end of the amplification product using a single-strand DNA ligase.

5. The method according to claim 1, wherein the adapter comprises a DNA molecular tag.

6. The method according to claim 1, wherein the index primer comprises entire or part of complementary sequence of the adapter.

7. The method according to claim 1, further comprising treating the DNA fragment with a type II restriction endonuclease before the step of treating the DNA fragment with sodium bisulfite.

8. The method according to claim 7, wherein the type II restriction endonuclease is selected from the group consisting of HpaII/MspI, SmaI/XmaI, BamHI and HpaII.

9. The method according to claim 1, further comprising: cyclizing the adapter-attached single-strand DNA fragment before the step of preparing the DNA sequencing library.

10. The method according to claim 9, wherein the adapter-attached single-strand DNA fragment is cyclized using a highly efficient cyclization agent.

11. The method according to claim 10, wherein the highly efficient cyclization agent is a single-strand DNA circligase.

12. The method according to claim 9, wherein, 5′ end of the adapter-attached DNA fragment is phosphorylated and 3′ end of the adapter-attached DNA fragment is dephosphorylated before the step of cyclizing the adapter-attached single-strand DNA.

13. The method according to claim 1, wherein the DNA fragment is cleaved to obtain cleaved DNA fragments with a size of 0.01-5 kb, 0.1-5 kb, 0.1-1 kb, 1-2 kb, 2-3 kb, 3-4 kb, 4-5 kb, 0.2-0.4 kb, 0.5-1 kb, 0.1-0.5 kb, 0.01-0.5 kb, 0.01-0.4 kb, 0.01-0.3 kb, 0.01-0.25 kb, 0.02-0.25 kb, 0.05-0.3 kb or 0.05-0.25 kb before the step of treating the DNA fragment with sodium bisulfite, wherein the cleaved DNA fragments contains the specific genome regions.

14. The method according to claim 9, wherein the step of preparing the DNA sequencing library comprises amplifying the cyclized single-strand DNA fragment with the index primer after the step of cyclizing the adapter-attached single-strand DNA fragment, and obtaining the DNA sequencing library comprising a DNA amplification product containing the specific genome regions.

15. The method according to claim 14, wherein the amplification step uses inverse PCR amplification.

16. The method according to claim 14, wherein the amplification step uses rolling circle amplification.

17. The method according to claim 1, wherein the sequencing step comprises using high throughput DNA sequencing technology to determine the sequence of the DNA sequencing library containing the specific genome regions.

18. The method according to claim 1, wherein the step of preparing the DNA sequencing library further comprises: enriching specific amplification product using an oligonucleotide probe after the amplification step.

19. The methods according to claim 1, wherein the DNA contained in the sample comprises cell-free DNA, preferably, the cell-free DNA also comprising circulating tumor DNA.

20. Kit for detecting methylation within specific genome regions in a DNA fragment from a sample containing DNA, comprising:
sodium bisulfite, which can be used to convert unmethylated cytosine in the DNA fragment into uracil;
a single-strand DNA ligating agent, which can be used to ligate a single-strand DNA fragment with an adapter; and
a library preparing agent, which can be used to amplify the single-strand DNA fragment containing specific genome regions.

21. The kit according to claim 20, wherein the single-strand DNA ligating agent comprises the adapter, a single-strand DNA ligase, and a reacting solution.

22. The kit according to claim 20, further comprises a linear amplification agent, wherein the linear amplification agent comprising a linear amplification primer, the linear amplification primer comprises entire or part of complementary sequence of the adapter attached to one end of the single-strand DNA fragment.

23. The kit according to claim 20, further comprises a cleaving agent, which can be used to cleave the DNA fragment into cleaved DNA fragments with size of 0.01-5 kb, 0.1-5 kb, 0.1-1 kb, 1-2 kb, 2-3 kb, 3-4 kb, 4-5 kb, 0.2-0.4 kb, 0.5-1 kb, 0.1-0.5 kb, 0.01-0.5 kb, 0.01-0.4 kb, 0.01-0.3 kb, 0.01-0.25 kb, 0.02-0.25 kb, 0.05-0.3 kb or 0.05-0.25 kb before sodium bisulfite treatment.

24. The kit according to claim 20, further comprises a sequencing agent.

25. The kit according to claim 20, wherein the library preparing agent comprises an index primer.

26. The kit according to claim 20, further comprises a cyclization agent, which can be used to cyclize single-strand DNA fragment.

27. The kit according to claim 26, wherein the cyclization agent is a highly efficient cyclization agent.

28. The kit according to claim 27, wherein the highly efficient cyclization agent is a single-strand DNA circligase.

29. The kit according to claim 26, wherein the cyclizing agent further comprises T4 PNK, which can be used to phosphorylate 5′ end and dephosphorylate 3′ end of a DNA fragment.

30. The kit according to claim 26, wherein the library preparing agent comprises an agent for use in inverse PCR amplification or an agent for use in rolling circle amplification.

31. Use of a single-strand DNA ligating agent in preparation of a kit for detecting methylation within specific genome regions in a DNA fragment from a sample containing DNA, wherein the detection comprises the following steps:
treating the DNA fragment containing specific genome regions with sodium bisulfite, wherein the treatment with sodium bisulfite converts unmethylated cytosine in the DNA fragment into uracil, and obtaining a single-strand DNA fragment;
attaching adapter to one end or both ends of the single-strand DNA fragment using the single-strand DNA ligating agent;
preparing a DNA sequencing library containing the specific genome regions by amplifying the adapter attached single-strand DNA fragment with index primer; and
sequencing the DNA sequencing library.

32. The use according to claim 31, wherein the detection further comprises denaturing the DNA fragment treated with sodium bisulfite.

33. The use according to claim 31, wherein adapter is attached to both ends of the single-strand DNA fragment via single-strand DNA ligase.

34. The use according to claim 31, wherein using single-strand DNA ligase to attach adapter to 3′ end of the single-strand DNA fragment, linearly amplifying the single-strand DNA fragment with adapter attached on 3′ end to obtain a amplification product, and using single-strand DNA ligase to attach adapter to 3′ end of the amplification product.

35. The use according to claim 31, wherein the detection further comprises: cyclizing the adapter attached single-strand DNA fragment using a highly efficient cyclizing agent, and amplifying the cyclized single-strand DNA fragment after the cyclizing step, wherein the sequencing process determines the sequence of the amplification product.

36. The use according to claim 35, wherein the detection further comprises: before cyclizing the single-strand DNA fragment, phosphorylating 5′ end and dephosphorylating 3′ end of the DNA fragment.

37. The use according to claim 31, wherein the detection further comprises: before the sequencing step, enriching the amplification product containing specific genome regions using oligonucleotide probe.
